(19) United States
(12) Patent Application Publication
(10) Pub. No.: US 2006/0276550 A1
(43) Pub. Date: Dec. 7, 2006

(54) Topical compositions of ketamine and butamben and methods of their use

(76) Inventor: Dileep Bhagwat, Ardsley, NY (US)

Correspondence Address: JONES DAY, 51 Louisiana Avenue N.W., Washington, DC, 20001-2113, US

(21) Appl. No.: 11/143,652

(22) Filed: Jun. 3, 2005

(51) Int. Cl. A61K 31/137

(52) U.S. Cl. 514/650

(57) ABSTRACT

Disclosed are pharmaceutical compositions comprising ketamine and butamben. The compositions are useful in the treatment of pain.

### FIELD OF THE INVENTION

The present invention relates to compositions containing ketamine and butamben and their use in treating pain.

### BACKGROUND OF THE INVENTION

Pain results from the noxious stimulation of nerve endings. Nociceptive pain is caused by noxious stimulation of nociceptors (e.g., a needle stick or skin pinch), which then transmit impulses over intact neural pathways to the spinal neurons and then to the brain. Neuropathic pain is caused by damage to neural structures, such as damage to peripheral nerve endings or nociceptors, which become extremely sensitive to stimulation and can generate impulses in the absence of stimulation (e.g., herpes zoster pain after the rash has healed). Peripheral nerve damage can lead to pathological states where there is a reduction in pain threshold (i.e., allodynia), an increased response to noxious stimuli (hyperalgesia), or an increased response duration (persistent pain). See Goodman & Gilman's The Pharmacological Basis of Therapeutics (Joel G. Hardman et al. eds., 9th ed. 1996); and Harrison's Principles of Internal Medicine 53-58 (Anthony S. Fauci et al. eds., 14th ed. 1998).

Some types of pain can be treated locally by topically administering a local anesthetic directly to the painful area to block the nociceptive mechanistic pathway. Local anesthetics prevent the generation and conduction of nociceptive nerve impulses. Thus, for example, a local anesthetic can be injected intradermally (non-systemic injection within the skin) or topically applied at the pain area. Advantages of topical local-anesthetic administration over systemic administration of pain relievers include decrease or preclusion of side effects, improved patient compliance, and reversible action (i.e., the action can be reversed by removing the anesthetic from the application site). Transdermal and Topical Drug Deliver Systems 33-112 (Tapash K. Ghosh et al. eds., 1997).

A variety of drug classes have local anesthetic properties. Traditional local anesthetics, or sodium-channel blockers, such as butamben, reportedly prevent the generation and conduction of nerve impulses by decreasing or preventing the large transient increase in the permeability of excitable membranes to sodium cations. Other agents with local-anesthetic properties include analgesics, such as non-steroidal anti-inflammatories (“NSAIDs”). See, e.g., id. at 87-93; see also U.S. Pat. No. 5,948,389 and C. Stein and A. Yassouridis Pain 71: 119 (1997).

Some N-methyl-D-aspartate (“NMDA”) receptor antagonists, such as ketamine, also have local-aesthetic properties. See, e.g., U.S. Pat. No. 5,817,699. Ketamine is reportedly useful for treating sympathetic mediated pain, myofascial pain, temporomandibular joint (TMJ) pain, osteoarthritis and sacroiliac joint (SIJ) pain, as well. See U.S. Pat. No. 6,017,961. Recently, it has been reported that the combination of the NMDA receptor antagonist ketamine with the sodium channel blocker butamben has a faster and greater analgesic onset than when ketamine or butamben are administered alone. Id.

Even though topical local anesthetic administration to intact skin is routinely used to treat minor indications, it has not found significant use for treating more severe nociceptive and neuropathic pain because it is difficult to achieve significant concentrations through the skin barrier. Because of the skin's drug-permeation resistance, as little as about one percent, and usually no more than about 15 percent of a drug in a topical formulation is bioavailable. Transdermal and Topical Drug Delivery, supra, at 7. Another problem with topical administration of pain relievers is the stability of the composition. Emulsions of local-anesthetics are typically unstable, and phase separation can occur during shipment and storage. Furthermore, many topical local-anesthetic compositions suffer from oxidative instability. For example, lecithin compositions are routinely used as bases for topical local aesthetic compositions, but are highly oxidatively unstable. American Pharmacology Association, Handbook of Pharmaceutical Excipients 292 (Arthur H. Kibbe ed., 3d ed. 2000).

In sum, the potential for some topical anesthetics to be used for the treatment of pain is hampered by their instability and poor skin-penetration properties. Thus, there is a need for stable topical local anesthetic compositions with good skin-penetration properties and stability.

### SUMMARY OF THE INVENTION

In one embodiment, this invention relates to a pharmaceutical composition comprising ketamine, butamben, an emollient, a humectant, a preservative, a non-ionic emulsifier, an anti-foaming agent and water.

In another embodiment, the invention relates to a pharmaceutical composition comprising ketamine, butamben, cetyl alcohol, isopropyl myristate, white petrolatum, sorbitol 70% solution in water, propylparaben, methylparaben, glyceryl stearate, PEG-100 stearate, simethicone and water.

Other embodiments of the invention encompass methods of using the composition described herein for the treatment of pain.

### DETAILED DESCRIPTION OF THE PREFERRED EMBODIMENTS

One embodiment of the invention is pharmaceutical composition comprising a therapeutically effective amount of ketamine and butamben, an emollient, a humectant, a preservative, a non-ionic emulsifier, a lubricant or anti-foaming agent and water. The pharmaceutical composition may contain ketamine and/or butamben as a free base, a pharmaceutically acceptable salt, a solvate (e.g., a hydrate), a complex (e.g., hydrate, solvate, and clathrate), a prodrug, or any stereoisomeric forms or mixtures of stereoisomeric forms (e.g., geometrical isomers, enantiomers, diastereomers, racemates, or mixtures thereof).

When topically administered to a mammal (e.g., a human, dog or a cat), compositions of the invention can induce local anesthesia and thereby treat, ameliorate, or prevent pain. Advantageously, pharmaceutical compositions of the invention are stable both physically (e.g., resistant to phase separation) and chemically (e.g., resistant to oxidation).

As used herein, a “therapeutically effective amount” of ketamine or butamben means the amount of ketamine or butamben required to induce a local-anesthetic effect sufficient to treat or ameliorate pain in a mammal.

As used herein, the term “mammal” means any mammal, including, but not limited to humans; pets, such as dogs and cats; farm mammals, such as horses, cows, pigs, and sheep; and laboratory animals, such as monkeys, guinea pigs, rats, and mice. Preferably, the mammal is a human.

The term “topical composition” means a pharmaceutical composition designed for topical administration and containing a pharmaceutical.

As used herein, the phrase “intradermally-acceptable” means any pharmaceutical, excipient or other component of a topical formulation that is safe or approved for intradermal or topical administration in mammals.

The phrase “pharmaceutically acceptable salt(s),” as used herein includes, but is not limited to, salts of acidic or basic groups that may be present in the compounds of the preferred embodiments of the invention.

Ketamine and butamben are basic in nature and are capable of forming a wide variety of salts with various inorganic and organic acids. The acids that may be used to prepare pharmaceutically acceptable salts of such basic compounds are those that form salts comprising pharmacologically acceptable anions including, but not limited to, acetate, benzenesulfonate, benzoate, bicarbonate, bitartrate, bromide, calcium edetate, camsylate, carbonate, chloride, bromide, iodide, citrate, dihydrochloride, edetate, edisylate, estolate, esylate, fumarate, gluceptate, gluconate, glutamate, glycollylarsanilate, hexylresorcinate, hydrabamine, hydroxynaphthoate, isethionate, lactate, lactobionate, malate, maleate, mandelate, mesylate, methylsulfate, muscate, napsylate, nitrate, panthothenate, phosphate/diphosphate, polygalacturonate, salicylate, stearate, succinate, sulfate, tannate, tartrate, teoclate, triethiodide, and pamoate (i.e., 1,1′-methylene-bis-(2-hydroxy-3-naphthoate)). Ketamine and butamben have an amino moiety and also can form pharmaceutically acceptable salts with various amino acids, in addition to the acids mentioned above.

In a preferred embodiment of the present invention, ketamine is in the form of ketamine hydrochloride.

As used herein and unless otherwise indicated, the term “prodrug” means a derivative of a compound that can hydrolyze, oxidize, or otherwise react under biological conditions (in vitro or in vivo) to afford ketamine or butamben. Examples of prodrugs include, but are not limited to, derivatives of compounds of the invention that comprise biohydrolyzable moieties such as biohydrolyzable amides, biohydrolyzable esters, biohydrolyzable carbamates, biohydrolyzable carbonates, biohydrolyzable ureides, and biohydrolyzable phosphate analogs. Other examples of prodrugs include derivatives of compounds of the invention that comprise —NO, —NO₂, —ONO, or —ONO₂ moieties. Prodrugs can typically be prepared using well-known methods, such as those described in Burger's Medicinal Chemistry and Drug Discovery, 172-178, 949-982 (Manfred E. Wolff ed., 5th ed. 1995) and Design of Prodrugs (H. Bundgaard ed. 1985).

As used herein and unless otherwise indicated, the terms “biohydrolyzable amide,” “biohydrolyzable ester,” “biohydrolyzable carbamate,” “biohydrolyzable carbonate,” “biohydrolyzable ureide,” “biohydrolyzable phosphate” mean an amide, ester, carbamate, carbonate, ureide, or phosphate, respectively, of a compound that either: 1) does not interfere with the biological activity of the compound but can confer upon that compound advantageous properties in vivo, such as uptake, duration of action, or onset of action; or 2) is biologically inactive but is converted in vivo to the biologically active compound. Examples of biohydrolyzable esters include, but are not limited to, lower alkyl esters, lower acyloxyalkyl esters (such as acetoxylmethyl, acetoxyethyl, aminocarbonyloxymethyl, pivaloyloxymethyl, and pivaloyloxyethyl esters), lactonyl esters (such as phthalidyl and thiophthalidyl esters), lower alkoxyacyloxyalkyl esters (such as methoxycarbonyloxymethyl, ethoxycarbonyloxyethyl and isopropoxycarbonyloxyethyl esters), alkoxyalkyl esters, choline esters, and acylamino alkyl esters (such as acetamidomethyl esters). Examples of biohydrolyzable amides include, but are not limited to, lower alkyl amides, α-amino acid amides, alkoxyacyl amides, and alkylaminoalkylcarbonyl amides. Examples of biohydrolyzable carbamates include, but are not limited to, lower alkylamines, substituted ethylenediamines, aminoacids, hydroxyalkylamines, heterocyclic and heteroaromatic amines, and polyether amines.

### A. Ketamine-Butamben Compositions

The amount of ketamine and butamben used in pharmaceutical compositions of the invention can vary. The skilled artisan will recognize that dosages and concentrations can be optimized according to routine experiments using well-known pain models, for example, those described in J. Sawynok et al., Pain 82: 149 (1999) and J. Sawynok et al. Pain 80: 45 (1999).

In general, the amount of ketamine in pharmaceutical compositions of the invention is within the range of from about 0.1 percent to about 20 percent by weight, from about 1 percent to about 10 percent by weight, or from about 2 percent to about 8 percent by weight.

In general, the amount of butamben in pharmaceutical compositions of the invention is within the range of from about 0.1 percent to about 15 percent by weight, from about 1 percent to about 8 percent by weight, or from about 2 percent to about 6 percent by weight.

In general, the ratio of ketamine to butamben in compositions of the invention is in the range of about 3:1 to about 1:3. In any event, the skilled artisan will recognize that the amount of ketamine and butamben should be sufficient to alleviate pain, but should be low enough to avoid causing adverse effects, such as nausea, vomiting, elevated blood pressure and pulse and arrythmia.

In one embodiment, pharmaceutical compositions of the invention comprise one or more emollients and one or more humectants. The pharmaceutical compositions may optionally contain one or more skin protectants. Examples of emollients include, but are not limited to, cetyl alcohol, isopropyl myristate, white petrolatum, cholesterol or linoleic acid, or mixtures thereof. Examples of humectants include, but are not limited to, glycerin, sodium lactate, sorbitol, polyethylene glycols (e.g., PEG-400), urea, propylene glycol, 1,3-butylene glycol, ethanol, and isopropanol, or mixtures thereof. Examples of skin protectants include, but are not limited to, vitamin E oil, allatoin, glycerin, zinc oxide, vitamins A, B (e.g., biotin and pantothenic acid), C, E, F, H, and P, and esters thereof. Specific emollients are white petrolatum, isopropyl myristate, and cetyl alcohol. A specific humectant is sorbitol, preferably, as a 70% aqueous solution.

In one embodiment, pharmaceutical compositions of the invention comprise one or more emollients in an amount from about 1% to about 25% by weight, from about 3% to about 15% by weight, or from about 3% to about 12% by weight.

In one embodiment, pharmaceutical compositions of the invention comprise one or more humectants in an amount from about 1% to about 15% by weight, from about 2% to about 10% by weight, or from about 4% to about 10% by weight.

Pharmaceutical compositions of the invention may comprise one or more preservatives. Typical amounts of preservatives can range from about 0.01% to about 2% by weight, from about 0.01% to about 0.5% by weight, or from about 0.02% by weight to about 0.25% by weight.

In some instances, it is also advantageous to include one or more antioxidants to preserve the medicaments and excipients present in the compositions. Some medicaments and excipients are oxygen labile and can undergo oxidation. Thus, certain compositions of the invention comprise an antioxidant in an amount of from about 0.001% to about 1% by weight, or from about 0.01% to about 0.5% by weight.

Examples of preservatives include, but are not limited to, quaternary amines, such as quaternium 15, benzalkonium chloride, cetrimide, benzethonium chloride; and imidizolidinyl urea; organic acids, such as sorbic acid, p-hydroxybenzoic acid, and benzoic acid; parabens, such as methyl paraben and propyl paraben; alcohols, such as benzyl alcohol and isopropyl alcohol; phenols, such as triclosan, chlorhexidine, and thimerosal; hydantoin derivatives; chloromethylthiazoline; methylisothiazoline; phenoxethol; hexetidine; chlorohexydingluconate; and imidazolidinylurea. Specific preservatives are methyl paraben, propyl paraben, and mixtures thereof.

Examples of antioxidants include, but are not limited to, ascorbic acid and its esters, sodium bisulfite, sodium metabisulfite, thiourea, butylated hydroxytoluene, butylated hydroxyanisole, tocopherols, alkyl gallates, and chelating agents like EDTA and citric acid.

Specific pharmaceutical compositions of the invention comprise one or more non-ionic emulsifiers. Non-ionic emulsifiers can be present in various amounts, for example, from about 2% to about 15% by weight, from about 5% to about 10% by weight, or from about 7% to about 9% by weight.

Examples of non-ionic emulsifiers include, but are not limited to, sorbitan monolaurate, sorbitan monostearate, glyceryl stearate, polyoxyl 50 stearate, polysorbate 60, polyoxyethylene stearate, PEG 20 sobitan monolaurate, PEG 20 sobitan monopalmitate, PEG 20 sorbitan monostearate, PEG 20 sobitan monooleate, PEG 20 sobitan trioleate, PEG 8 stearate, PEG 40 stearate, PEG 100 stearate, and other PEG stearates; PEG 4 lauryl ether, PEG 21 stearyl ether, PEG 2 oleyl ether. A specific emulsifier is PEG 100 stearate.

Specific pharmaceutical compositions of the invention comprise one or more anti-foaming agents, which may facilitate manufacture. Anti-foaming agents dissipate foam by destabilizing the air-liquid interface and allow liquid to drain away from air pockets. The amount of anti-foaming agent can be, for example, from about 0.01% to about 1% by weight, from about 0.1% to about 0.5% by weight, or from about 0.1% to about 0.2% by weight. Examples of anti-foaming agents include simethicone, dimethicone, ethanol, and ether. A specific anti-foaming agent is simethicone.

In another embodiment, pharmaceutical compositions of the invention comprise one or more skin penetration enhancers. Skin penetration enhancers can be present in various amounts, for example, from about 0.1% to about 20% by weight or from about 2% to about 5% by weight.

Skin penetration enhancers can be included to optimize transfer of ketamine and butamben through the stratum corneum and into the dermis to provide a local effect. For a discussion of use of penetration enhancers in topical formulations see generally, Percutaneous Penetration Enhancers (Eric W. Smith & Howard I. Maibach eds. 1995); Ghosh, T. K. et al. Pharm. Tech. 17: 72 (1993); Ghosh, T. K. et al. Pharm. Tech. 17: 62 (1993); Ghosh, T. K. et al. Pharm. Tech. 17: 68 (1993), all of which citations are hereby incorporated herein by reference. The penetration enhancer should be pharmacologically inert, non-toxic, and non-allergenic, have rapid and reversible onset of action, and be compatible with the compositions of the preferred embodiments of the invention.

Examples of skin penetration enhancers include, but are not limited to, caprylic and capric triglycerides, transcutol P, ethyl alcohol, isopropyl alcohol, lauryl alcohol, salicylic acid, octolyphenylpolyethylene glycol, polyethylene glycol 400, propylene glycol, N-decylmethylsulfoxide, DMSO and the azacyclo compounds, as disclosed in U.S. Pat. Nos. 4,755,535; 4,801,586; 4,808,414; and 4,920,101, all of which patents are incorporated herein by reference.

Specific pharmaceutical compositions of the invention comprise ketamine, butamben, cetyl alcohol, isopropyl myristate, white petrolatum, sorbitol 70% solution in water, propylparaben, methylparaben, glyceryl stearate, PEG-100 stearate, simethicone and water.

In one embodiment, the composition comprises about 5% by weight ketamine, about 2% by weight butamben, about 9% by weight cetyl alcohol, about 12% by weight isopropyl myristate, about 6% by weight white petrolatum, about 10% by weight sorbitol 70% solution in water, about 0.2% by weight methylparaben, about 2% by weight propylparaben, about 6% by weight glyceryl stearate, and about 0.1% by weight simethicone.

Specific pharmaceutical compositions of the present invention can further comprise one or more additional local anesthetics in addition to ketamine and butamben. Additional local anesthetics can be present in various amounts, for example, from about 0.1% to about 20% by weight, from about 5% to about 20% by weight, or from about 5% to about 10% by weight.

Examples of local anesthetics suitable for use with the invention include sodium-channel blockers, opioids and non-steroidal anti-inflammatories (“NSAIDs”).

Examples of sodium-channel blockers include, but are not limited to, ambucaine, amolanone, amylcaine, benoxinate, benzocaine, betoxycaine, biphenamine, bupivacaine, butacaine, butamben, butanilicaine, butethamine, butoxycaine, carticaine, chloroprocaine, cocaethylene, cocaine, cyclomethycaine, dibucaine, dimethisoquin, dimethocaine, diperodon, dyclonine, ecogonidine, ecogonine, euprocin, fenalcomine, formocaine, hexylcaine, hydroxyteteracaine, isobutyl p-aminobenzoate, leucinocaine, levoxadrol, lidocaine, mepivacaine, meprylcaine, metabutoxycaine, methyl chloride, myrtecaine, naepaine, octacaine, orthocaine, oxethazaine, parenthoxycaine, phenacaine, phenol, piperocaine, piridocaine, polidocanol, pramoxine, prilocaine, procaine, propanocaine, proparacaine, propipocaine, propoxycaine, pseudococaine, pyrrocaine, ropivacaine, salicyl alcohol, tetracaine, tolycaine, trimecaine, zolamine, or pharmaceutically-acceptable salts or mixtures thereof. Specific sodium-channel blockers, include lidocaine, procaine, bupivacaine, prilocaine, mepivacaine, etidocaine, ropivacaine, dibucaine, and pharmaceutically-acceptable salts and mixtures thereof.

Examples of opioids include, but are not limited to, alfentanil, allylprodine, alphaprodine, anileridine, benzylmorphine, benzitramide, nor-binaltorphimine, bremazocine, buprenorphine, butorphanol, clonitazene, codeine; selective receptor antagonists, such as D-Phe-Cys-Tyr-D-Trp-Orn-Thr-Pen-Thr-NH₂(CTOP), ala(2)-mephe(4)-gly(5)-ol-enkephalin (DAMGO), and c-[d-Pen², d Pen⁵]enkephalin (DPDPE); desomorphine, dextromoramide, dezocine, diampromide, dihydrocodeine, dihydrocodeine enol acetate, dihydromorphine, dimenoxadol, dimepheptanol, dimethylthiambutene, dioxaphetyl butyrate, dipipanone, diprenorphine, eptazocine, ethoheptazine, ethylketocyclazocine, ethylmethylthiambutene, etonitazene, etorphine, fentanyl, hydrocodone, hydromorphone, hydroxypethidine, isomethadone, ketobemidone, levorphanol, lofentanil, loperamide, meperidine, meptazinol, metazocaine, methadone, metopon, morphine, myrophine, nalbuphine, naltrindole, benzoylhydrazone, naltrexone, narceine, nicomorphine, norlevorphanol, normethadone, normorphine, norpipanone, opium, oxycodone, oxymorphone, papaveretum, papaverine, pentazocine, phenadoxone, henazocine, phenoperidine, piminodine, pirtramide, proheptazine, promedol, propiram, propoxyphene, remifentanil, spiradoline, sufentanil, tilidine, U50,488 (trans-3,4-dichloro-N-methyl-N-(2-(1-pyrrolidinyl)cyclohexyl)-benzeneacetamide) and U69,593 (N-methyl-N-(7-(1-pyrrolidinyl)-1-oxaspiro(4,5)dec-8-yl)benzeneacetamide), amiphenazole, cyclazocine, levallorphan, nalmefene, nalorphine, naloxone, and naltrexone or pharmaceutically-acceptable salts and mixtures thereof.

Specific opioids include morphine, loperamide, and loperamide derivatives such as those disclosed in U.S. Pat. Nos. 5,763,445; 5,981,513; 5,869,521; 5,744,458; 5,760,023; 5,798,093; 5,849,762; 5,811,078; 6,004,964; 5,962,477; 5,688,955; 5,888,494; 5,646,151; and 5,667,773 or pharmaceutically-acceptable salts and mixtures thereof, all of which patents are incorporated herein by reference.

Examples of NSAIDS include, but are not limited to, acetylsalicylic acid, ketoprofen, ibuprofen, piroxicam, diclofenac, indomethacin, and ketorolac.

Some compositions of the invention comprise an agent to prolong the local-anesthetic effect, such as, but not limited to, a glucocorticosteroid (see, e.g., U.S. Pat. No. 5,922,340, incorporated herein by reference) or a vasoconstrictor, such as a catecholamine.

Specific compositions of the invention comprise one or more additional ingredients, such as one or more thickening agents, medicinal agents or pharmaceuticals, bioadhesive polymers, inert carriers, lipid absorbents, viscosity stabilizers, chelating agents, buffers, anti-fading agents, stabilizers, moisture absorbents, fragrances, colorants, film-forming materials, and refatting agents. One of skill in the art will readily be able to choose such additional excipients based on the physical and chemical properties desired in the final topical formulation. The skilled artisan will also recognize that one excipient, including all of the specific excipients disclosed above, may have multiple functions and properties (e.g., some emollients may also act as emulsifiers).

Specific compositions of the invention comprise one or more thickening agents. Thickening agents can be present in various amounts, for example, from about 1% to 10% by weight or from about 2% to about 5% by weight.

Examples of thickening agents include, but are not limited to, triethanolamine, cellulose, hydroxypropyl cellulose, methyl cellulose, polyethylene glycol, sodium carboxymethyl cellulose, polyethylene oxide, xanthan gum, guar gum, agar, carrageenan gum, gelatin, karaya, pectin, and locust-bean gum, aliginic acid, carbomer (e.g., bentonite carbomer), povidone, and tragacanth.

Specific compositions of the invention comprise medicinal agents, in addition to ketamine and butamben, or their pharmaceutically acceptable salts. One of skill in the art can readily choose a medicinal agent to incorporate into the compositions of the invention and its appropriate concentration depending on the indication and desired effect. Examples of medicinal agents include, but not limited to, antifungals such as ciclopirox, chloroxylenol, triacetin, sulconazole, nystatin, undecylenic acid, tolnaftate, miconizole, clotrimazole, oxiconazole, griseofulvin, econazole, ketoconozole, and amphotericin B; antibiotics, such as mupirocin, erthromycin, clindamycin, gentamicin, polymyxin, bacitracin, and silver sulfadiazine; antiseptics, such as iodine, povidine-iodine, benzalkonium chloride, benzoic acid, chlorhexidine, nitrofurazone, benzoyl peroxide, hydrogen peroxide, hexachlorophene, phenol, resorcinol, and cetylpyridinium chloride; and anti-inflammatories, such as hydrocortisone, prednisone, triamcinolone, betamethasone, and dexamethasone.

Compositions of the invention can include one or more bioadhesive polymers. Examples of bioadhesive polymers include, but are not limited to, pectin, alginic acid, chitosan, hyaluronic acid, polysorbates, such as polysorbate-20, -21, -40, -60, -61, -65, -80, -81, -85; poly(ethyleneglycol), such as PEG-7, -14, -16, -18, -55, -90, -100, -135, -180, -4, -240, -6, -8, -9, -10, -12, -20, or -32; oligosaccharides and polysaccharides, such as gellan, carrageenan, xanthan gum, gum Arabic, and dextran; cellulose esters and cellulose ethers; modified cellulose polymers, such as carboxymethylcellulose, hydroxyethylcellulose, hydroxypropyl methylcellulose, hydroxyethyl ethylcellulose; polyether polymers and oligomers, such as polyoxyethylene; condensation products of poly(ethyleneoxide) with various reactive hydrogen containing compounds having long hydrophobic chains (e.g., aliphatic chains of about 12 to 20 carbon atoms), for example, condensation products of poly(ethylene oxide) with fatty acids, fatty alcohols, fatty amides, polyhydric alcohols; polyether compounds, such as poly(methyl vinyl ether), polyoxypropylene of less than 10 repeating units; polyether compounds, such as block copolymers of ethylene oxide and propylene oxide; mixtures of block copolymers of ethylene oxide and propylene oxide with other excipients, for example, pluronic lethicin organogel; poly(vinyl alcohol); polyacrylamide; hydrolyzed polyacrylamide; poly(vinyl pyrrolidone); poly(methacrylic acid); poly(acrylic acid) or crosslinked polyacrylic acid, such as carbomer, i.e., a homopolymer of acrylic acid crosslinked with either an allyl ether of pentaerythritol, an allyl ether of sucrose, or an allyl ether of propylene (e.g., Acrisint™400, 410, or 430 commercially available from 3V Inc. Weehawkin, N.J.); Orabase™(i.e., a mixture of gelatin, pectin and sodium carboxymethyl cellulose in a plasticized hydrocarbon gel, commercially available from Hoyt Laboratories, Needham, Mass.); Carafate™(sulfated sucrose and aluminum hydroxide, commercially available from Marion Laboratories, Inc., Kansas City, Mo.). Specific block copolymers are ethylene oxide and propylene oxide.

Compositions of the invention may be prepared according to standard methods, well known in the art, for preparing oil-in-water emulsions for topical administration. For example, the methods recited in Remington: The Science and Practice of Pharmacy 289 (Alfonso R. Gennaro ed., 19th ed. 1995), incorporated herein by reference, can be used. An exemplary composition is described in the Example section below.

For example, the components can be separated into those that are water soluble and those that are oil soluble. The water-soluble components can be mixed together in one vessel to form a solution, and the oil-soluble components can be mixed together in a separate vessel and heated (e.g., 70° C. to 80° C.) to form a solution. The two solutions can then be mixed and the mixture allowed to cool. This method requires nothing more than two beakers and a heating apparatus. Homogenation is achieved using a high-shear rate blender or other suitable apparatus. The appropriate droplet size is achieved by standard adjustment of the shear rate during high-speed mixing followed by droplet size analysis. See Remington: The Science and Practice of Pharmacy, supra, at 282-283 and Allen & Terence, Particle Size Measurement 483 (4th ed. 1990), both of which citations are incorporated herein by reference. Suitable equipment and methods for preparing emulsions and compositions of the preferred embodiments of the invention, such as high-shear rate blenders. See Remington: The Science and Practice of Pharmacy, supra, at 1509-1515, incorporated herein by reference. Methods for preparation of emulsions for topical administration, suitable for preparing compositions of the present invention, are also described in Bernard Idson, Pharmaceutical Emulsions in Pharmaceutical Dosage Forms: Disperse Systems 199 (Herbert A. Lieberman et al. eds. 1988), incorporated herein by reference.

Specific compositions of the invention can be packaged and stored according to well-known methods. See, e.g., the packaging procedures described in Remington: The Science and Practice of Pharmacy, supra, at 390-391, hereby incorporated herein by reference. If desired, the compositions of the preferred embodiments of the invention can be sterilized according to well-known methods. Id. at 1463-1486.

### B. Methods of Use of Ketamine-Butamben Compositions

Pharmaceutical compositions of the present invention can be used to treat or prevent various types of pain, including, but not limited to, associated with, or induced by, the following diseases, trauma, or conditions: general neuropathic conditions, such as peripheral neuropathy, phantom pain, reflex-sympathetic dystrophy, causalgia, syringomyelia, and painful scar; neuralgia; back pain; diabetic neuropathy; alcoholic neuropathy; metabolic neuropathy; inflammatory neuropathy; chemotherapy-induced neuropathy, herpetic neuralgias; traumatic odontalgia; endodontic odontalgia; thoracic-outlet syndrome; cervical, thoracic, or lumbar radiculopathies with nerve compression; cancer with nerve invasion; traumatic-avulsion injuries; mastectomy, thoracotomy pain; spinal-cord-injury; stroke; abdominal-cutaneous nerve entrapments; tumors of neural tissues; arachnoiditis; stump pain; fibromyalgia; regional sprains or strains; myofascial pain; psoriatic arthropathy; polyarteritis nodosa; osteomyelitis; burns involving nerve damage; AIDS-related pain syndromes; connective tissue disorders, such as systemic lupus erythematosis, systemic sclerosis, polymyositis, and dermatomyositis; and inflammatory conditions, such as acute inflammation (e.g., trauma, surgery and infection) and chronic inflammation (e.g., arthritis and gout).

Compositions of the invention can be topically administered to intact skin by a medical professional or by the patient by simple mechanical rubbing into the application site. In applying these compositions to the skin, for maximum effectiveness and increased absorption, the area to which the composition is to be administered is first cleansed with an astringent, such as a standard commercial antiseptic or alcohol. The area is then allowed to dry for a few seconds. Next, the composition can be rubbed on to the complete target area of the skin (the painful area) and gently, but firmly, massaged in with the fingertips until all visible gel or cream has been absorbed.

After application of compositions of the invention, the application site can be covered with a dressing. The term “dressing,” as used herein, means a covering designed to protect a previously applied drug formulation. “Dressing” includes coverings such as a bandage, which may be porous or non-porous and various inert coverings, e.g., a plastic film wrap or other non-absorbent film. The term “dressing” also encompasses non-woven or woven coverings, particularly elastomeric coverings, which allow for heat and vapor transport. These dressings allow for cooling of the pain site, which provides for greater comfort. In another embodiment, a composition of the preferred embodiments of the invention can be incorporated into a dressing, which dressing is then applied to the skin or painful area.

In one embodiment, compositions of the invention can be contained in a patch that is applied on or adjacent to the area of skin to be treated. As used herein a “patch” comprises at least a composition of the invention and a covering layer, such that, the patch can be placed over the area of skin to be treated. In one embodiment, the patch can be designed to maximize drug delivery through the stratum corneum and into the epidermis or dermis, and to minimize absorption into the circulatory system, reduce lag time, promote uniform absorption, and reduce mechanical rub-off. The patch components can resemble the viscoelastic properties of the skin and conform to the skin during movement to prevent undue shear and delamination.

Patches have advantages over conventional methods of administration. One advantage is that the dose is controlled by the surface area of the patch. Other advantages of patches are constant rate of administration, longer duration of action (e.g., the ability to adhere to the skin for 1, 3, 7 days or longer); improved patient compliance, non-invasive dosing, and reversible action (i.e., the patch can simply be removed).

Examples of patches suitable for use with the present invention include (1) the matrix-type patch; (2) the reservoir-type patch; (3) the multi-laminate drug-in-adhesive type patch; (4) the monolithic drug-in-adhesive type patch; and (5) hydrogel patch. See generally Transdermal and Topical Drug Delivery Systems, supra, at 249-297, incorporated herein by reference. These patches are well known in the art and commercially available.

In one embodiment, compositions of the invention are contained in a reservoir-type patch. The reservoir-type patch is characterized by a backing film coated with an adhesive and a reservoir compartment comprising the composition. See, e.g., U.S. Pat. No. 4,615,699, incorporated herein by reference. The adhesive coated backing layer can extend around the reservoir's boundaries to provide a concentric seal with the skin and hold the reservoir adjacent to the skin.

In another embodiment, a composition of the invention is contained in a drug-in-adhesive or hydrogel patch. The monolithic drug-in-adhesive patch design is characterized by the inclusion of the drug formulation in the skin contacting adhesive layer, a backing film and preferably, a release liner. The adhesive functions both to release the anesthetic and adhere the anesthetic matrix to the skin. The drug-in-adhesive system does not require an adhesive overlay and thus the patch size is minimized. Also, drug-in-adhesive type patches are thin and comfortable. See, e.g., U.S. Pat. No. 4,751,087, incorporated herein by reference.

The multi-laminate drug-in-adhesive patch design further incorporates additional semi-permeable membrane between two distinct drug-in-adhesive layers or multiple drug-in-adhesive layers under a single backing film. See Peterson, T. A. and Dreyer, S. J. Proceed. Intern. Symp. Control Rel Bioact. Mater. 21: 477-478, incorporated herein by reference.

Semi permeable membranes, useful with the reservoir or multi-laminate patch, include thin non-porous ethylene vinyl acetate films or thin microporous films of polyethylene employed in microlaminate solid state reservoir patches.

Adhesives for use with the drug-in-adhesive type patches are well known in the art and selection is readily accomplished by the skilled artisan. Four basic types commonly used are polyisobutylenes, silicones, acrylics, and hydrogels. Adhesives useful in the present invention can function under a wide range of conditions, such as, high and low humidity, bathing, and sweating. In one embodiment, the adhesive is a composition based on natural or synthetic rubber, polyacrylate, polyvinylacetate, polybutylacrylate, polymethylacrylate, polydimethylsiloxane, and hydrogels (e.g., high molecular weight polyvinylpyrrolidone, oligomeric polyethylene oxide, or a mixture thereof). Specific adhesives are, e.g., polyacrylate and hydrogels. For example, the hydrogel can be electron-beam cross-linked polyvinylpyrrolidone (“PVP”) where the PVP is of an average molecular weight of about 500,000 Daltons to about 2,000,000 Daltons, or about 900,000 Daltons to about 1,500,000 Daltons. Exemplary PVP-hydrogels for use in the invention are described in Published PCT Application No. WO93/10163; U.S. Pat. No. 4,989,607; European Patent No. 0107376; D. Darwis Radiat. Phys. Chem. 42: 907 (1993); and Olgun Guven & Murat Sen Polymer 32: 2491 (1991), all of which citations are incorporated herein by reference.

Suitable release liners include, but are not limited to, occlusive, opaque, and clear polyester films with a thin coating of pressure sensitive release liner (e.g., silicone-fluorsilicone, and perfluorcarbon based polymers).

Backing films may be occlusive or permeable, and are derived from synthetic polymers like polyolefin oils polyester, polyethylene, polyvinylidine chloride, and polyurethane or from natural materials like cotton, and wool. Occlusive backing films, such as synthetic polyesters, result in hydration of the outer layers of the stratum corneum while non-occlusive backings allow the area to breath (i.e., promote water vapor transmission from the skin surface). More preferably the backing film is an occlusive polyolefin foil (Alevo, Dreieich, Germany). The polyolefin foil is preferably about 0.6 to about 1 mm thick.

Certain compositions of the invention constitute from about 0.5 percent to about 40 percent by weight, or from about 10 percent to about 30 percent, or from about 15 percent to about 25 percent, or from about 18 percent to about 22 percent by weight of the patch.

Patches that can be used with compositions of the invention can be manufactured, packaged, stored and labeled according to standard procedures. See, e.g., the procedures described in Bova et al., Product Development and Technology Transfer for Transdermal Therapeutic Systems in Transdermal Controlled Systemic Medications 379-396 (Y. W. Chien ed. 1987); J. W. Dohner, Development of Processes and Equipment for Rate Controlled Transdermal Therapeutic Systems in Transdermal Controlled Systemic Medications 349-364 (Y. W. Chien ed. 1987); H-M Wolf et al., Development of Processes and Technology for Adhesive-Type Transdermal Therapeutic Systems in Transdermal Controlled Systemic Medications 365-378 (Y. W. Chien ed. 1987), all of which citations are incorporated herein by reference.

Selection of an appropriate dosage for the application site can be an important consideration. The rate of intradermal anesthetic administration from the topical formulation or patch is a function of skin permeability, and skin permeability has been shown to vary between anatomical sites depending on the thickness of the stratum corneum. For example, the permeability, in general, increases in order from planter foot arch, lateral ankle, palm, ventral forearm, dorsal forearm, back, chest, thigh, abdomen, scalp, axilla, forehead, and scrotum. See R. C. Wester. & H. I. Maibach Regional variation in Percutaneous Absorption in Percutaneous Absorption, Mechanism, Methodology, Drug Delivery 111-119 (R. L. Bronaugh & H. I. Maibach eds., 2nd ed. 1989), incorporated herein by reference. Of course, the dosages and dosing frequency will be determined by the skilled artisan and will depend upon many factors such as application site and size and the severity of the indication.

With gels, creams, and ointments, typically about one to four applications are required per day. Generally, about 0.1 g/cm² of skin area to about 10 g/cm², preferably 1 g/cm² to about 5 g/cm² of a composition of the invention is administered to and around the application site. The skilled artisan will realize, however, that the amount of composition of the invention that must be administered, will depend on the surface area of the affected area to which the composition is to be administered. After administration, if desired, the area can be covered with a dressing.

When a patch is used to administer compositions of the invention, the dosage to achieve pain relief may be determined by the active surface area of the medicated portion of the patch in direct contact with the skin. Several dosage strengths are advantageous, depending upon the severity of the wound. In general, a physician can begin dosing with a low or intermediate strength patch and then, depending upon the effectiveness, adjust the dosage up or down by prescribing a patch of higher or lower active concentration or a patch of larger or smaller surface area, or, in some cases, multiple patches.

### C. Examples

An example of a pharmaceutical composition of the present invention is described in Table 1, below.

TABLE 1

| | Example 1 | Example 2 |
|---|---|---|
| Ingredient | % w/w | % w/w |
| Ketamine HCl | 4.61 | 9.23 |
| Butamben | 2.0 | 4.0 |
| Methylparaben | 0.20 | 0.20 |
| Propylparaben | 0.02 | 0.02 |
| 70% Sorbitol solution | 9.76 | 9.76 |
| PEG-100 stearate | 8.94 | 8.94 |
| Cetyl alcohol | 3.58 | 3.58 |
| Isopropyl myristate | 12.1 | 12.1 |
| Glyceryl stearate | 6.05 | 6.05 |
| White Petrolatum | 5.78 | 5.78 |
| Simethicone | 0.1 | 0.1 |
| Purified Water QS | 100 | 100 |

Specific compositions of the invention comprising ketamine and butamben can be formulated as follows:

Purified water, 70% sorbitol solution and PEG-100 stearate are stirred and subsequently heated in a suitable steam jacketed mixing tank or equivalent, thereby yielding a water phase. The temperature of the mixture is maintained in the range 70°-80° C. When a clear solution is formed, methylparaben and ketamine HCl are added to the mixture. The mixture is stirred until all ingredients are dissolved.

In a separate suitable steam jacketed mixing tank or equivalent, cetyl alcohol, isopropyl myristate, glycerol stearate and white petrolatum are heated and stirred thereby yielding an oil phase. The temperature of the mixture is maintained in the range 70°-80° C. The mixture is heated and stirred until a homogenous liquid results. Propylparaben and butamben are then added and stirring is continued until the propylparaben is completely dissolved.

The oil phase is slowly added to the water phase (both phases being in the temperature range of about 70°-80° C.) with continuous stirring. The mixture may then be homogenized using a high shear homogenizer or an equivalent thereof. The resulting emulsion is mixed and then cooled. When the emulsion temperature drops below 40° C., simethicone is added while mixing, and the mixture is allowed to cool to room temperature.

All reagents used in the above example are commercially available from standard sources. For example, ketamine hydrochloride can be purchased from Medisca, Inc., Plattsburg, N.Y.

From the foregoing description, one skilled in the art can easily ascertain the essential characteristics of this invention, and without departing from the spirit and scope thereof, can make various changes and modifications of the preferred embodiments of the invention to adapt it to various usages and conditions without undue experimentation. All patents, patent applications and publications cited herein are incorporated by reference in their entirety.

What is claimed is:

1. A pharmaceutical composition comprising ketamine, butamben, an emollient, a humectant, a preservative, a non-ionic emulsifier, an anti-foaming agent and water.

2. The composition of claim 1, wherein the emollient is cetyl alcohol, isopropyl myristate, white petrolatum, cholesterol or linoleic acid.

3. The composition of claim 1, wherein the humectant is glycerin, sodium lactate, PEG-400, propylene glycol or sorbitol 70% solution in water.

4. The composition of claim 1, wherein the preservative is propylparaben or methylparaben.

5. The composition of claim 1, wherein the non-ionic emulsifier is glyceryl stearate, PEG-100 stearate, sorbitan mono stearate, polyoxyl 50 stearate, or polysorbate 60.

6. The composition of claim 1, wherein the anti-foaming agent is simethicone, dimethicone, ethanol, or ether.

7. The composition of claim 1, further comprising a skin penetration enhancer.

8. The composition of claim 7, wherein the skin penetration enhancer is a caprylic triglyceride, capric triglyceride or urea.

9. The composition of claim 1 further comprising a thickener.

10. The composition of claim 9, wherein the thickener is carbomer, triethanolamine or xantham gum.

11. The composition of claim 2, wherein the emollient is white petrolatum, isopropyl myristate, or cetyl alcohol.

12. The composition of claim 3, wherein the humectant is 70% aqueous sorbitol solution.

13. The composition of claim 5, wherein the non-ionic emulsifiers are glyceryl stearate and PEG 100 stearate.

14. The composition of claim 6, wherein the anti-foaming agent is simethicone.

15. The composition of claim 1, wherein from about 0.1 percent to about 20 percent of its weight is ketamine or a pharmaceutically acceptable salt thereof.

16. The composition of claim 1, wherein from about 0.1 percent to about 15 percent of its weight is butamben or a pharmaceutically acceptable salt thereof.

17. The composition of claim 1, wherein from about 1% to about 25% of its weight is an emollient.

18. The composition of claim 1, wherein from about 1% to about 15% of its weight is a humectant.

19. The composition of claim 1, wherein from about 0.01% to about 2% of its weight is a preservative.

20. The composition of claim 1, wherein from about 2% to about 15% of its weight is a non-ionic emulsifier.

21. The composition of claim 1, wherein from about 0.01% to about 1% of its weight is an anti-foaming agent.

22. The composition of claim 15, wherein about 5% of its weight is ketamine.

23. The composition of claim 16, wherein about 2% of its weight is butamben.

24. The composition of claim 17, wherein about 12% of its weight is an emollient.

25. The composition of claim 18, wherein about 10% of its weight is a humectant.

26. The composition of claim 19, wherein about 2% of its weight is a preservative.

27. The composition of claim 20, wherein about 9% of its weight is a non-ionic emulsifier.

28. The composition of claim 21, wherein about 0.1% of its weight is an anti-foaming agent.

29. A pharmaceutical composition comprising ketamine, butamben, cetyl alcohol, isopropyl myristate, white petrolatum, sorbitol 70% solution in water, propylparaben, methylparaben, glyceryl stearate, PEG-100 stearate, simethicone and water.

30. A method of treating pain comprising administering to a subject in need thereof, a therapeutically effective amount of a composition of claim 1.

31. A method of treating pain comprising administering to a subject in need thereof, a therapeutically effective amount of a composition of claim 29.

32. The method of one of claims 30 or 31, wherein the pain is neuropathic or sympathetic pain.

33. The method of one of claims 30 or 31, wherein the pain is peripheral neuropathy, phantom pain, reflex-sympathetic dystrophy, causalgia, syringomyelia, painful scar, diabetic neuropathy; alcoholic neuropathy; metabolic neuropathy; inflammatory neuropathy; chemotherapy-induced neuropathy, herpetic neuralgias, traumatic odontalgia, endodontic odontalgia, thoracic-outlet syndrome, cervical, thoracic, or lumbar radiculopathies with nerve compression, cancer with nerve invasion, traumatic-avulsion injuries, mastectomy, thoracotomy pain, spinal-cord-injury, stroke, abdominal-cutaneous nerve entrapments, tumors of neural tissues, arachnoiditis, stump pain, fibromyalgia, regional sprains or strains, myofascial pain, psoriatic arthropathy, polyarteritis nodosa, osteomyelitis, burns involving nerve damage, AIDS-related pain syndromes, systemic lupus erythematosis, systemic sclerosis, polymyositis, dermatomyositis, acute inflammation, or chronic inflammation.
